SEQUENCE LISTING (15 sequences)

SEQ ID NO: 1 - DNA, 34 bp, Artificial sequence
  Synthetic
ttaagcttgccaccatggctgttctgggcatcac

SEQ ID NO: 2 - DNA, 30 bp, Artificial sequence
  Synthetic
ttggtaccttacgagcggggaatgacacag

SEQ ID NO: 3 - DNA, 37 bp, Artificial sequence
  Synthetic
tttaagcttgccaccatggtgagcaagggcgaggagc

SEQ ID NO: 4 - DNA, 31 bp, Artificial sequence
  Synthetic
tttctgcagttacttgtacagctcgtccatg

SEQ ID NO: 5 - DNA, 22 bp, Artificial sequence
  Synthetic
cttgatatacttggatgatggc

SEQ ID NO: 6 - DNA, 18 bp, Artificial sequence
  Synthetic
agccacgcacatttagga

SEQ ID NO: 7 - DNA, 20 bp, Artificial sequence
  Synthetic
tgctgtgatgctgtttgttg

SEQ ID NO: 8 - DNA, 17 bp, Artificial sequence
  Synthetic
ccggaaacaaacaacga

SEQ ID NO: 9 - DNA, 20 bp, Artificial sequence
  Synthetic
acgactctcggcaacggata

SEQ ID NO: 10 - DNA, 21 bp, Artificial sequence
  Synthetic
ttgcgttcaaagactcgatgg

SEQ ID NO: 11 - DNA, 19 bp, Artificial sequence
  Synthetic
aaggaggacggcaacatcc

SEQ ID NO: 12 - DNA, 23 bp, Artificial sequence
  Synthetic
aagttcaccttgatgccgttctt

SEQ ID NO: 13 - DNA, 20 bp, Artificial sequence
  Synthetic
ttgtggttctgcacggctac

SEQ ID NO: 14 - DNA, 20 bp, Artificial sequence
  Synthetic
cttccaggtgggtccattgt

SEQ ID NO: 15 - protein, 493 aa, Rabbit
MAVLGITVALLGWMVILLFISVWKQIHSSWNLPPGPFPLPIIGNLLQLDLKDIPKSFGRLAERFGPVFTVYLGSRRVVVLHGYKAVREMLLNHKNEFSGRGEIPAFREFKDKGIIFNNGPTWKDTRRFSLTTLRDYGMGKQGNEDRIQKEAHFLLEELRKTQGQPFDPTFVIGCTPFNVIAKILFNDRFDYKDKQALRLMSLFNENFYLLSTPWLQVYNNFSNYLQYMPGSHRKVIKNVSEIKEYTLARVKEHHKSLDPSCPRDFIDSLLIEMEKDKHSTEPLYTLENIAVTVADMFFAGTETTSTTLRYGLLILLKHPEIEEKLHEEIDRVIGPSRMPSVRDRVQMPYMDAVVHEIQRFIDLVPSNLPHEATRDTTFQGYVIPKGTVVIPTLDSLLYDKQEFPDPEKFKPEHFLNEEGKFKYSDYFKPFSAGKRVCVGEGLARMELFLLLSAILQHFNLKPLVDPEDIDLRNITVGFGRVPPRYKLCVIPRS

(19) United States
(12) Patent Application Publication
(10) Pub. No.: US 2022/0081694 A1
(43) Pub. Date: Mar. 17, 2022

(54) INDOOR AIR POLLUTANT DEGRADATION BY GENETICALLY MODIFIED PLANTS

(71) Applicant: University of Washington, Seattle, WA (US)

(72) Inventors: Long Zhang, Seattle, WA (US); Stuart E. Strand, Seattle, WA (US)

(73) Assignee: University of Washington, Seattle, WA (US)

(21) Appl. No.: 17/422,977

(22) PCT Filed: Dec. 18, 2019
(86) PCT No.: PCT/US2019/067265

§ 371 Date: Jul. 14, 2021

Related U.S. Application Data
(60) Provisional application No. 62/792,726, filed on Jan. 15, 2019.

(51) Int. Cl. C12N 15/82; B01D 53/85; B01D 53/72; B01D 53/70; A01G 9/24

(52) U.S. Cl. CPC C12N 15/8242; B01D 53/85; B01D 53/72; B01D 2257/708; A01G 9/246; B01D 2257/2064; B01D 2257/7027; B01D 53/70

(57) ABSTRACT

A genetically modified houseplant capable of reducing levels of volatile organic carcinogenic compounds, such as formaldehyde, benzene, and chloroform, in the indoor air in urban homes of developed countries is disclosed. The plant expresses a detoxifying transgene, mammalian cytochrome P450 2e, and has shown sufficient detoxifying activity against benzene and chloroform. Air purifying biofilters utilizing the plants and methods of their use are also disclosed.

### CROSS-REFERENCE TO RELATED APPLICATION

This application claims the benefit of U.S. Provisional Patent Application No. 62/792,726, filed Jan. 15, 2019, which is incorporated herein by reference in its entirety.

### STATEMENT OF GOVERNMENT LICENSE RIGHTS

This invention was made with Government support under CBET-1438266 awarded by the National Science Foundation and the National Institute of Environmental Health Sciences Grant No. 2P42-ES004696-19. The Government has certain rights in the invention.

### FIELD OF THE INVENTION

The invention relates to transgenic houseplants capable of removing volatile organic carcinogens (VOCs) from indoor air and biofilters comprising such transgenic plants.

### STATEMENT REGARDING SEQUENCE LISTING

The sequence listing associated with this application is provided in text format in lieu of a paper copy and is hereby incorporated by reference into the specification. The name of the text file containing the sequence listing is 70586_Seq_Final_2019-12-16.txt. The text file is 6.92 KB; was created on Dec. 16, 2019; and is being submitted via EFS-Web with the filing of the specification.

### BACKGROUND

Household air is more polluted than office air and school air, and those who spend much of their time at home, such as children and home workers, receive a proportionately higher dose of home air carcinogens than the general population. Infants are particularly susceptible to indoor air pollution due to their low body weight and continuous exposure to indoor air. The highest risk volatile organic carcinogens (VOCs) are benzene, formaldehyde, 1,3-butadiene, carbon tetrachloride, acetaldehyde, 1,4-dichlorobenzene (PDCB), naphthalene, perchloroethylene, chloroform, and ethylene dichloride. VOCs that exceeded acute exposure standards are acrolein and formaldehyde (during cooking) and chloroform (during showering).

Some sources of these chemicals can be eliminated or reduced. For example, PDCB could be greatly reduced by eliminating products containing it from the home. Formaldehyde in household air can be reduced by changing construction and upholstery material compositions, but formaldehyde is also emitted from other sources, including cooking, which are not easily eliminated. Other carcinogens with multiple sources are more difficult to eliminate, such as benzene, which originates from fuel storage in attached garages, outside air, and environmental tobacco smoke.

Physical-chemical methods for VOC removal include adsorption on activated carbon, activated alumina, zeolites or other surfaces and photocatalytic oxidation. Adsorption methods are not well suited for formaldehyde and other polar compounds. Low molecular weight compounds may be desorbed in competition with higher molecular weight pollutants. Adsorption methods are not destructive, and the sorbents must be periodically regenerated, usually remotely using energy intensive methods. Low temperature in-place methods achieved energy efficient regeneration but would require exterior ducting. Oxidation methods use photocatalysized redox destruction of VOCs on catalytic materials, such as TiO₂. Photocatalytic oxidation methods result in complete mineralization of most pollutants, but they are ineffective with chlorinated VOCs such as chloroform. Further, photocatalytic oxidation methods may introduce ozone into the home air, and they are energy intensive.

Indoor plants have been widely touted as having the ability to remove air pollutants from indoor air. This approach is known as the “green liver” concept and is a central idea of the field of phytoremediation, the use of plants to remove xenobiotic pollutants from the environment. Early studies of air detoxification by household plants found that formaldehyde was removed from the air of chambers containing spider plants. Other researchers reported that soil or water alone could explain the removal. Subsequently, controlled pure culture plant experiments showed that plants can assimilate and metabolize formaldehyde from the air. However, the formaldehyde uptake rate through the leaf surface of typical house plants appears to be insufficient to remove formaldehyde from a typical room without an excessive number of plants. Several studies have found that common plants can remove VOCs such as formaldehyde and benzene from air, but those studies produced highly variable estimates of the rate that a particular plant species removes a given pollutant from air. The concentrations used in these tests were several orders of magnitude greater that those typical of home air (e.g., 1-7 μg m⁻³).

These conflicting data notwithstanding, plants do have many attractive features as a platform for metabolism of organic pollutants. Unlike most bacteria, cultivated plants have excess energy available to support cometabolic catalysis. Plants have high surface areas that facilitate mass transfer of trace gases from the air. Plants are self-sustaining and do not require the high maintenance typical of bacterial systems. There is certainty of the genetic and enzymatic composition of the cultivated plant compared to a soil bacterial community.

Plants have been genetically modified to overexpress native plant formaldehyde dehydrogenase activity, but the rate of formaldehyde removal was increased by only 25% over unmodified plants. Expression in transformed tobacco plants of the transgene for formaldehyde dehydrogenase, faldh, from Brevibacillus brevis increased formaldehyde removal by three-fold. However, to date, no detoxifying genes have been expressed in houseplants.

Thus, a need exists for plants that can be grown indoors as houseplants and provide efficient phytoremediation of indoor air by metabolizing VOCs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, the expression cassette further comprises a second polynucleotide sequence encoding a visual selection marker, a third polynucleotide sequence encoding a positive selection marker, a fourth polynucleotide encoding an aldehyde dehydrogenase, or a combination thereof. In some embodiments, each of the first, second, third, and fourth polynucleotide sequences is operably linked to a suitable promoter functional in the transgenic houseplant.

In some embodiments, the transgenic houseplant is transgenic Epipremnum aureum.

In some embodiments, the mammalian cytochrome protein is Cytochrome P450 or a variant or a homolog thereof with at least about 90% amino acid sequence homologous thereto, such as rabbit Cytochrome P450 2E1 or a variant or a homolog thereof with at least about 90% amino acid sequence homologous thereto.

In some embodiments, each of the first, second, third, and fourth polynucleotide sequences is codon optimized for plants, for example, monocotyledon plants.

In some embodiments, the visual selection marker is a fluorescent protein, such as mGFP-ER, eGFP, or a variant or a homolog thereof with at least about 90% amino acid sequence homologous thereto. In some embodiments, the positive selection marker is Hygromycin B phosphotransferase or a variant or a homolog thereof with at least about 90% amino acid sequence homologous thereto. In some embodiments, the fourth polynucleotide sequence encoding an aldehyde dehydrogenase enzyme such as formaldehyde dehydrogenase or a variant or a homolog thereof with at least about 90% amino acid sequence homologous thereto.

In some embodiments, the transgenic houseplant is capable of removing a volatile organic compound selected from the group consisting of 1,4-dichlorobenzene, benzene, 1,3-butadiene, formaldehyde, acetaldehyde, naphthalene, acrylonitrile, carbon tetrachloride, dichlorobromomethane, dibromochloromethane, bromoform, trichloroethylene, vinyl chloride, methyl chloroform, cis-1,2-dichloroethylene, chloroform, and combinations thereof.

In a second aspect, provided herein is a method of phytoremediation comprising contacting a transgenic houseplant disclosed herein with air comprising a volatile organic compound vapor, thereby causing the volatile organic compound to be removed from the air. In some embodiments, contacting the air comprising a volatile organic compound (VOC) vapor with a transgenic houseplant disclosed herein results in reduction of the concentration of the VOC in the air.

In a third aspect, provided herein is an air purifying biofilter comprising one or more transgenic houseplant disclosed herein. In some embodiments, the air purifying biofilter comprises: (a) a planting structure comprising one or more transgenic houseplants disclosed herein and (b) an air flow device coupled to the planting structure and adapted to maintain airflow around the plants. In some embodiments, the planting structure is a green wall. In some embodiments, the air purifying biofilter system further comprises an enclosure surrounding the one or more transgenic houseplants. In some embodiments, the air purifying biofilter further comprises a means for self-irrigation, a light source, or a combination thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the structure of binary vector pRCS2-2E1-EGFP used to transform pothos ivy. In this Figure, T35s is terminator of CaMV 35s gene; hpt is hygromycin phosphotransferase gene, provides hygromycin resistance; OsActin is promoter of actin gene of Oryza sativa; Tmas is terminator of manopine synthase gene; 2e1 is cytochrome P450 2E1 gene from rabbit; ZmUbi is promoter of ubiquitin of Zea mays; PvUbi is promoter of ubiquitin gene of Panicum virgatum (switchgrass); egfp is enhanced green fluorescent protein; Trbc is terminator of rubisco small subunit gene; LB is left border of T-DNA region; and RB is right border of T-DNA region.

FIGS. 2A-2D show transformation of pothos ivy with 2e1 gene via Agrobacterium infection. Leaf discs and fragments of petiole of pothos ivy were infected with EHA105 harboring pRCS2-2E1-EGFP. The explants were cultured on somatic embryo induction medium with hygromycin at 20 mg L⁻¹ for selection. (2A) Callus developed from explants after 3-4 months screening. (2B) Hygromycin resistant callus was transferred to regeneration medium supplied with hygromycin at 20 mg L⁻¹ for 2-4 months to induce development of new plantlets. (2C) Regenerated plants were transferred to MS medium with hygromycin at 20 mg L⁻¹ for rooting and growth. (2D) PCR- and RT-PCR-positive transformed plants were cultured on callus induction medium with hygromycin for callus induction and propagation.

FIG. 3 is a graph of a transcript abundance measured using quantitative RT-PCR on pothos ivy lines transformed with 2e1 and egfp genes. The y-axis shows values that were normalized to the pothos ivy 5.8s rRNA gene and relative to the level of VD1 (n=3±SE). Letters indicate means that were not significantly different (p=0.05, ANOVA).

FIGS. 4A and 4B show observation of EGFP signal in the epidermal cells of pothos ivy clone VD3 using fluorescence microscopy. The green fluorescence signal of EGFP was observed in cytosol in the epidermal cells of leaf of pothos ivy clone VD3 (4A). The emissions in the wild-type pothos ivy (4B) were due to autofluorescence.

FIG. 5 is a graph of uptake of benzene by 2e1-egfp-transformed pothos ivy grown in liquid culture. The concentration of benzene in headspace during eight-day culture of VD3 (2e1), wild-type plants (WT), and no-plant controls (NPC) is shown. N=4. Averages±SE.

FIG. 6 is a graph of uptake of chloroform by 2e1-egfp-transformed pothos ivy grown in liquid culture. The concentration of chloroform in headspace during eleven-day culture of VD3, wild-type plants (WT), and no-plant controls (NPC) are shown. N=4±SE.

FIG. 7 is a semi-log plot of the time course of benzene concentrations in batch incubation of transgenic clone VD3 of pothos ivy. R²=0.781. Slope=−0.249 d⁻¹, t statistic=−7.81, p=5×10⁻⁷.

FIG. 8 is a semi-log plot of the time course of benzene concentrations in batch incubation of wild-type pothos ivy. R²=0.299. Slope=−0.044 d⁻¹, t statistic=−2.69, p=0.015.

FIG. 9 is a semi-log plot of the time course of chloroform concentrations in batch incubation of transgenic clone VD3 of pothos ivy. R²=0.77. Slope=−0.578 d⁻¹, t statistic=−5.49, p=0.0004.

FIG. 10 is a semi-log plot of the time course of chloroform concentrations in batch incubation of wild-type pothos ivy. R²=0.23. Slope=−0.0.026 d⁻¹, t statistic=−1.32, p=0.22.

FIG. 11 shows a sequence of an exemplary mammalian cytochrome used in preparation of an exemplary transgenic houseplant (SEQ ID NO:15).

FIG. 12 is a photograph of an exemplary air purifying biofilter.

FIG. 13 shows a sequence encoding an exemplary FALDH that can be used in preparation of an exemplary transgenic houseplant (SEQ ID NO:16).

FIG. 14 shows a sequence of an exemplary FALDH protein (SEQ ID NO:18).

FIG. 15 shows a sequence encoding an exemplary ALDH1a1 that can be used in preparation of an exemplary transgenic houseplant (SEQ ID NO:16).

FIG. 16 shows a sequence of an exemplary ALDH1a1 protein (SEQ ID NO:18).

### DETAILED DESCRIPTION

The disclosure relates to genetically modified houseplants, houseplant parts, and houseplant cells and methods of their use in bioremediation of volatile organic carcinogens (VOCs). In one aspect, provided herein is genetically modified houseplant, i.e., transgenic houseplant, capable of reducing levels of VOCs, such as formaldehyde, benzene, and chloroform in surrounding air. The plant expresses a detoxifying transgene, e.g., a mammalian cytochrome P450 2e, and has sufficient detoxifying activity against volatile organic carcinogens such as benzene and chloroform.

In some embodiments, the transgenic houseplant disclosed herein is stably transformed with an expression cassette comprising a first polynucleotide sequence encoding a mammalian cytochrome protein, wherein the houseplant is capable of removing a volatile organic compound, such as a carcinogen, from air. As used herein, the terms “genetically modified” and “transgenic” are used interchangeably. In some embodiments, the mammalian cytochrome protein is Cytochrome P450, a variant thereof, or a homolog thereof with at least about 80%, at least about 85%, or at least about 90% amino acid sequence homologous thereto. In certain embodiments, the mammalian cytochrome protein is Cytochrome P450 2E1.

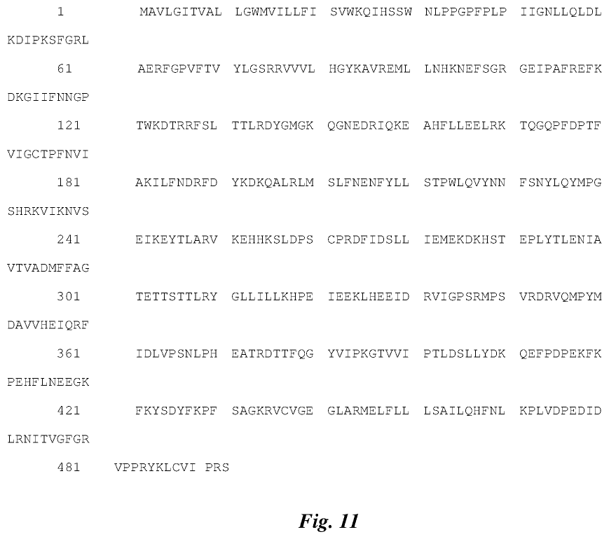

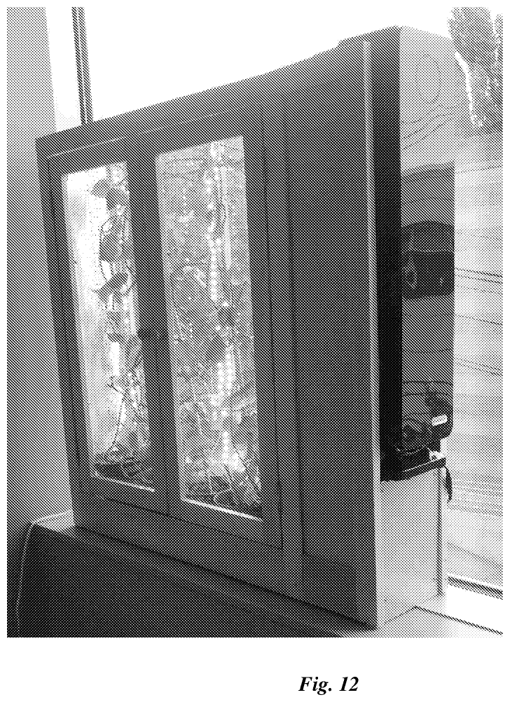

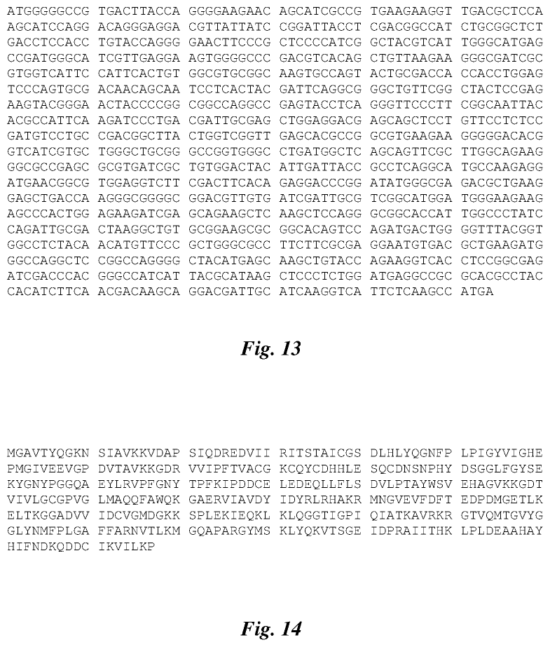

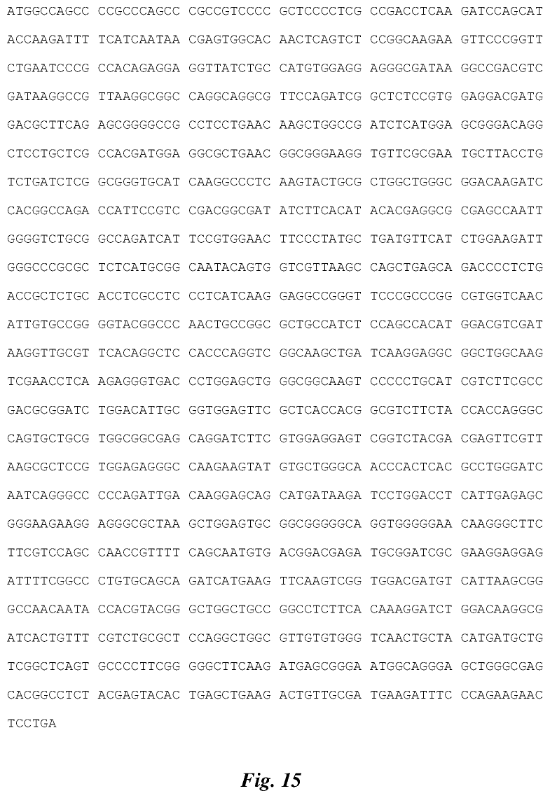

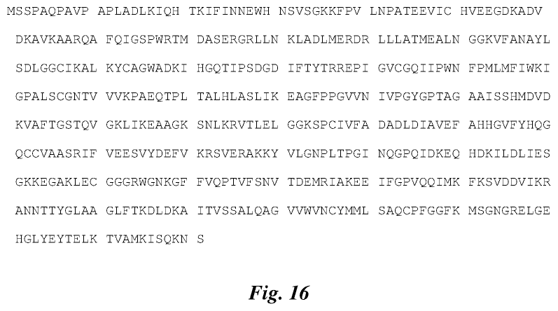

The mammalian cytochrome P450 2E1 (also referred to herein as 2E1) can oxidize a wide range of important VOCs that can be found in typical home air, such as benzene, chloroform, trichloroethylene, and carbon tetrachloride. The CYP2e1 (2e1) gene has been previously successfully introduced into trees, but no such modification of a houseplant has been proposed. Any mammalian cytochrome P450 2E1 can be used in the generation of the houseplant disclosed herein, for example, rabbit cytochrome P450 2E1. In some embodiments, the mammalian cytochrome has a sequence as shown in FIG. 11, a variant, or a homolog thereof with at least about 80%, at least about 85%, or at least about 90% amino acid sequence homologous thereto. In some embodiments, the mammalian cytochrome comprises SEQ ID NO: 15, a variant, or a homolog thereof with at least about 80%, at least about 85%, or at least about 90% amino acid sequence homologous thereto.

In some embodiments, the polynucleotide sequence encoding a mammalian cytochrome protein, for example, rabbit cytochrome P450 2E1, is codon optimized for plants. In certain embodiments, the polynucleotide sequence encoding a mammalian cytochrome protein is codon optimized for monocotyledon plants. In some embodiments, the first polynucleotide sequence encoding a mammalian cytochrome is operably linked to a first promoter functional in the houseplant. Any suitable promoters functional in the houseplant disclosed herein can be used in the transgenic houseplant disclosed herein. In some embodiments, a first promoter is a promoter functional in pothos ivy (Epipremnum aureum). In certain embodiments, the first promoter is a promoter disclosed by Zhao et al. (Zhao, J. T.; Li, Z. J. T.; Cui, J.; Henny, R. J.; Gray, D. J.; Xie, J. H.; Chen, J. J., Efficient somatic embryogenesis and Agrobacterium-mediated transformation of pothos (Epipremnum aureum) ‘Jade’. Plant Cell Tiss Org 2013, 114, (2), 237-247), which is incorporated herein by reference. Exemplary promoters suitable for inclusion in the expression cassette of the transgenic houseplant disclosed herein are described below in the Examples.

In some embodiments, the houseplant is Epipremnum aureum, commonly referred to as pothos ivy. Its other common names include pothos, devil's ivy, and variegated philodendron. Pothos ivy provides several advantages over other houseplants or other plants for the use in the generation of the transgenic houseplants disclosed herein: it is robust, grows well in low light, and does not flower in indoor cultivation or outdoors in the US or Canada, which is an advantage for biosafety considerations regarding the release of the transgenic plants into the environment.

In some embodiments of the transgenic houseplants, houseplant parts, and houseplant cells disclosed herein, the expression cassette further comprises a second polynucleotide sequence encoding a visual selection marker. Any suitable visual selection marker can be used. In certain embodiments, the visual selection marker is a fluorescent protein. Exemplary fluorescent proteins suitable for inclusion as a visual marker include green fluorescent proteins such as mGFP-ER and eGFP, their variants, and homologs thereof with at least about 80%, at least about 85%, or at least about 90% amino acid sequence homologous thereto.

Inclusion of a visual selection marker allows to distinguish the plant from wild type and thus to provide additional biosafety assurances by observing the unique fluorescence signature of the marker. Any suitable method of visualization can be used. For example, the transgenic plants disclosed herein can be monitored by simple visual observation or through the use of a detection device. Instrumentation and methods for detection and quantification of visual markers such as fluorescent proteins in plants is known in the art, for example, instrumentation and methods disclosed in Reginald J. Millwood, Hong S. Moon, and C. Neal Stewart Jr. Fluorescent Proteins in Transgenic Plants, Reviews in Fluorescence, 387-403 (2008), which is incorporated herein by reference.

In some embodiments, the polynucleotide sequence encoding a visual marker, for example, a green fluorescent protein, is codon optimized for plants. In certain embodiments, the second polynucleotide sequence encoding a visual marker is codon optimized for monocotyledon plants. In some embodiments, the second polynucleotide sequence is operably linked to a second promoter functional in the houseplant. Any suitable promoters functional in the houseplant disclosed herein can be used in the transgenic houseplant disclosed herein, such as those described above.

In certain embodiments, the expression cassette further comprises a third polynucleotide sequence encoding a positive selection marker. A positive selection marker is a gene that confers resistance to an agent that kills a wild type houseplant. Any suitable positive selection marker can be used. For example, in certain embodiments, the positive selection marker is a protein conferring antibiotic resistance to the houseplant. In certain embodiments, the positive selection marker is Hygromycin B phosphotransferase conferring resistance to hygromycin to the houseplant, for example, pothos ivy. In other embodiments, the positive selection marker is a gene conferring resistance to an herbicide, for example, the gene described in Thompson C J, Movva N R, Tizard R, et al. Characterization of the herbicide-resistance gene bar from Streptomyces hygroscopicus. EMBO J. 1987; 6(9):2519-23, the disclosure of which is incorporated herein by reference.

In some embodiments, the polynucleotide sequence encoding a positive selection marker, for example, Hygromycin B phosphotransferase, is codon optimized for plants, for example, for monocotyledon plants. In some embodiments, the third polynucleotide sequence is operably linked to a third promoter functional in the houseplant, such as the promoters described above.

In some embodiments, it is advantageous to have a transgenic houseplant expressing more than one detoxifying enzyme capable of removing VOCs from home air, for example, for removal of more than one chemical class of VOCs. Thus, in some embodiments, the transgenic houseplants described herein express a mammalian cytochrome such as 2e1 and one or more other detoxifying genes. For example, as described below in the Examples, the faldh gene can be stacked with 2e1 and/or other detoxifying genes in vectors that are used to genetically modify a houseplant, such as pothos ivy, resulting in houseplants that could degrade formaldehyde, one of the most important indoor air VOCs. Thus, in certain embodiments of the transgenic houseplant, houseplant part, or houseplant cell disclosed herein, the expression cassette further comprises a fourth polynucleotide sequence encoding a formaldehyde dehydrogenase, a variant, or a homolog thereof with at least about 80%, at least about 85%, or at least about 90% amino acid sequence homologous thereto. In some embodiments, the fourth polynucleotide sequence is operably linked to a fourth promoter functional in the houseplant. Any suitable promoters functional in the houseplant disclosed herein can be used in the transgenic houseplant disclosed herein, such as those promoters described above.

In some embodiments, provided herein is a houseplant cell or a houseplant portion stably transformed with an expression cassette comprising a first polynucleotide sequence encoding a mammalian cytochrome protein operably linked to a first promoter functional in the houseplant and a second polynucleotide sequence encoding a visual selection marker operably linked to a second promoter operable in the houseplant.

In yet another aspect, disclosed herein is a method of phytoremediation comprising contacting a transgenic houseplant of the disclosure with air comprising a volatile organic compound vapor or volatile organic carcinogen vapor, thereby causing the volatile organic compound to be removed from the air. In certain embodiments, the air is indoor air such as household air.

In some embodiments, the transgenic houseplant disclosed herein can remove or reduce concentration in air of any volatile organic carcinogen or volatile organic compound (VOC) oxidizable by a mammalian cytochrome. As used herein, the term “removing” a pollutant such as a VOC includes partial removal, e.g., reduction of concentration, and complete removal, e.g., below level detectable by methods typically used for analysis of such VOC. In some embodiments, the VOCs suitable for removal using the methods disclosed herein include aromatic compounds, unsaturated hydrocarbons, ketones, aldehydes, and halogenated hydrocarbons. Non-limiting examples of such VOCs include 1,4-dichlorobenzene, benzene, 1,3-butadiene, acetaldehyde, naphthalene, acrylonitrile, carbon tetrachloride, dichlorobromomethane, dibromochloromethane, bromoform, trichloroethylene, vinyl chloride, methyl chloroform, cis-1,2-dichloroethylene, chloroform, and combinations thereof.

In yet another embodiment provided herein is an air purifying biofilter comprising one or more transgenic houseplants of the disclosure, e.g., a transgenic houseplant stably transformed with an expression cassette comprising polynucleotide sequence encoding a mammalian cytochrome as described above. In some embodiments, the transgenic plants are Epipremnum aureum stably transformed with an expression cassette comprising polynucleotide sequence encoding a mammalian cytochrome as described above.

In order to maximize the phytoremediation potential of the transgenic houseplants disclosed herein, in some embodiments, it is advantageous to create or to increase mass air transfer around the transgenic houseplant, for example, move air over the leaf surfaces of the houseplant disclosed herein. In certain embodiments, the air purifying biofilter further comprises a means for increasing mass air flow over the leaf surfaces of the transgenic houseplant, i.e., an air flow device. In some embodiments, the air purifying biofilter comprises: (a) a plant receptacle comprising one or more transgenic houseplants disclosed herein and (2) an air flow device coupled to the plant receptacle and adapted to maintain airflow around the plant.

Any suitable means for increasing mass air flow can be included in the air purification units or biofilters disclosed herein, which include mechanical means (such as by operating an electric or manual fan). In some embodiments, the means can create a pressure or temperature differential, and the air flow increase can take place passively, for example, as a function of pressure differentials or temperature gradient present in the environment surrounding the transgenic houseplant.

In some embodiments, the air purifying unit comprises an enclosure that houses the one or more transgenic houseplants. In some embodiments, the air purifying unit is a green wall comprising one or more transgenic houseplants of the disclosure and a means for increasing mass air flow around the one or more plants. In some embodiments, a green wall is a vertical built structure is intentionally covered by vegetation and also can be referred to as a living wall or a vertical garden.

In some embodiments, the biofilter meets the standard used by the Association of Home Appliance Manufacturers (AHAM), for example, the standards for the rating of particulate biofilters or the “⅔ rule”: i.e., the clean air delivery rate (CADR, ft³/min, or m³/h) is greater than or equal to ⅔ times the room area, ft², assuming an 8 ft ceiling. Particulate filters are tested by the AHAM in a standard room containing 1008 ft³ (28.5 m³), with an 8 ft ceiling and an area of 126 ft² (11.7 m²). For this room, a filter that meets the ⅔ rule would have a CADR equal to ⅔ times 126 or 84 cfm (2.4 m³/min) or 0.67 cfm CADR per ft².

Thus, in some embodiments, the biofilters disclosed herein are capable of about 75% or greater, about 80% or greater, about 85% or greater, or about 90% or greater reduction in particulates in a room with exchange with exterior spaces equal to one room volume air change per hour. In some embodiments, substantially all particles with an average size of 2 μm or greater are removed from the air during the passage through the air purifying biofilter.

The following examples are provided for the purpose of illustrating, not limiting, the invention.

### Examples

### Materials and methods

Preparation of Pothos Ivy

Golden pothos ivy plants, obtained from a retail horticulture store, were grown under 50 μE m⁻² sec⁻¹ illumination with a 16 h day/8 h night cycle at 25° C. in a plant room. The stem fragments were excised, surface-sterilized with 15% sodium hypochlorite and then washed with sterile deionized water three times. The sterilized stem fragments were cultured on solid Murashige and Skoog's (MS) basic medium 25 in culture vessels. After 1-2 months culture under light, new leaves and roots developed from stems and these sterile plants were used for infection with engineered agrobacteria for genetic modification.

Vector Construction and Genetic Transformation

In order to genetically modify pothos ivy a genetic vector containing the transgenes 2e1, egfp, and hpt, each flanked by promoter and terminator sequences suitable for pothos ivy was constructed. The hpt gene coded for hygromycin B phosphotransferase, which confers resistance to hygromycin. Hygromycin was used to select for transformed cells since it kills wild-type pothos. These three genes were integrated into a transformation vector (“binary vector”) based on a system of cloning vectors called pSAT containing insertion sites for use with specific restriction enzymes (Chung, S. M.; Frankman, E. L.; Tzfira, T., A versatile vector system for multiple gene expression in plants. Trends in Plant Science 2005, 10, (8), 357-361). Then the binary vector was introduced into the modified Agrobacterium strain EHA105, which was used to infect pothos ivy callus cultures.

The rabbit cytochrome P450 2e1 gene was amplified by polymerase chain reaction (PCR) from the plasmid pSLD50-6 (the sequences of the primers, SEQ ID NOS 1-14 are listed in Table 1), a kind gift from S. L. Doty (University of Washington) and double digested with restriction enzymes HindIII and KpnI.

TABLE S1
The DNA sequences of primers used in preparing
exemplary vectors

| | | | Restriction |
|---|---|---|---|
| | SEQ | | recognition |
| | ID | | sequence |
| Application | NO | Sequence (5′-3′) | added (5′-3′) |
| Clone 2e1 | 1 | TTAAGCTTGCCACCATG | HindIII |
| gene | | GCTGTTCTGGGCATCAC | |
| | 2 | TTGGTACCTTACGAGCG | KpnI |
| | | GGGAATGACACAG | |
| Clone egfp | 3 | tttAAGCTTgccaccAT | HindIII |
| gene | | GGTGAGCAAGGGCGAGG | |
| | | AGC | |
| | 4 | tttCTGCAGTTACTTGT | PstI |
| | | ACAGCTCGTCCATG | |
| PCR to confirm | 5 | CTTGATATACTTGGATG | |
| the genetic | | ATGGC | |
| transformation | 6 | AGCCACGCACATTTAGG | |
| of pothos | | A | |
| | 7 | TGCTGTGATGCTGTTTG | |
| | | TTG | |
| | 8 | CCGGAAACAAACAACGA | |
| qRT-PCR | 9 | ACGACTCTCGGCAACGG | |
| | | ATA | |
| | 10 | TTGCGTTCAAAGACTCG | |
| | | ATGG | |
| | 11 | AAGGAGGACGGCAACAT | |
| | | CC | |
| | 12 | AAGTTCACCTTGATGCC | |
| | | GTTCTT | |
| | 13 | TTGTGGTTCTGCACGGC | |
| | | TAC | |
| | 14 | CTTCCAGGTGGGTCCAT | |
| | | TGT | |

Then 2e1 DNA was inserted into cloning vector pNSAT3a to produce pNSAT3a-2E1. After insertion into pNSAT3a, the 2e1 gene was integrated between promoter and terminator sequence to produce an expression cassette that drives the expression of 2e1 in plant cells. The egfp gene was cloned by PCR from vector pGH00.0126 (as described in Maximova, S. et al., Stable transformation of Theobroma cacao L. and influence of matrix attachment regions on GFP expression. Plant Cell Rep 2003, 21, (9), 872-83) and inserted into pNSAT6a as a HindIII-PstI fragment to produce pNSAT6a-EGFP. The expression cassettes of hpt, 2e1, and egfp genes were cut from pNSAT1a-HPT (as described in Zhang, L.; et al., Expression in grasses of multiple transgenes for degradation of munitions compounds on live-fire training ranges. Plant Biotechnol J 2017, 15, (5), 624-633), pNSAT3a-2E1, and pNSAT6a-EGFP vectors using restriction enzymes AscI, I-PpoI, and PI-PspI separately and inserted into the pRCS2 binary vector to produce pRCS2-2E1-EGFP.

The binary vector pRCS2-2E1-EGFP was transferred into Agrobacterium strain EHA105 by the freeze-thaw method (Chen, H.; Nelson, R. S.; Sherwood, J. L., Enhanced recovery of transformants of Agrobacterium tumefaciens after freeze-thaw transformation and drug selection. Biotechniques 1994, 16, (4), 664-8, 670) and the resulting strain, EHA105 (pRCS2-2E1-EGFP) was grown in LB medium (lysogeny broth) with 50 mg L−1 rifampicin, 100 mg L⁻¹ spectinomycin, and 300 mg L⁻¹ streptomycin for infection of pothos ivy. EHA105 (pRCS2-2E1-EGFP) was initiated in 100 mL LB medium with rifampicin at 50 mg L⁻¹, spectinomycin at 100 mg L⁻¹ and cultured over night at 28° C. on a rotary shaker at 200 rpm. The bacteria were centrifuged at 4,000 rpm for 10 min and resuspended in liquid E medium (MS medium with 2 mg L⁻¹ thidiazuron (TDZ) and 0.2 mg L⁻¹ 1-naphthaleneacetic acid (NAA)) with 100 μM acetosyringone (AS) and cultured under the same conditions until OD600 (absorbance of bacteria suspension at 600 nm) of 0.8-1.0 was reached.

The following method for transformation of pothos ivy was adapted from that of Zhao et al (Zhao, J. T., et al., Efficient somatic embryogenesis and Agrobacterium-mediated transformation of pothos (Epipremnum aureum) ‘Jade’. Plant Cell Tissue and Organ Culture, 2013. 114(2): p. 237-247). Leaf discs and petiole segments from sterile pothos plants were immersed in the agrobacterium culture at 25° C. for 20 min and then transferred to double-layered filter paper moistened with liquid E medium with AS at 100 μM in petri dish for 5-day coculture at 25° C. The leaf discs and petiole fragments were washed with sterile water and transferred to E medium with 100 mg L⁻¹ cefotaxime, 100 mg L⁻¹ carbenicillin (PhytoTechnology Laboratories) and 20 mg L⁻¹ hygromycin for screening. The explants were subcultured to fresh selection medium every three weeks.

After 2-3 months selection, the somatic embryos that developed from explants on selection medium were transferred to fresh medium for another month and then transferred to G medium (MS medium with 2 mg L⁻¹ 6-benzylaminopurine (6-BA) and 0.2 mg L⁻¹ NAA) with 100 mg L⁻¹ cefotaxime, 100 mg L⁻¹ carbenicillin and 20 mg L⁻¹ hygromycin and cultured under light for regeneration. After culture for two months, the regenerated plantlets were transferred to MS medium with 100 mg L⁻¹ cefotaxime, 100 mg L⁻¹ carbenicillin and 20 mg L⁻¹ hygromycin for rooting and growth, which required two additional months of culture.

Molecular Analysis of Transformed Plants

For polymerase chain reaction (PCR) analysis, the DNeasy plant mini kit (Qiagen, Valencia, Calif., USA) was used to purify DNA from hygromycin-resistant plants. PCR reactions were carried out with primers specific to 2e1 and egfp cassettes (as shown in Table 1).

Total RNA was extracted from the leaves of plants using the RNeasy plant mini kit (Qiagen, Valencia, Calif., USA). For real-time quantitative RT-PCR analysis, 1 μg of total RNA was transcribed to cDNA using M-MLV reverse transcriptase (Promega, Madison, Wis., USA). Real-time quantitative PCR was performed using the SensiFAST SYBR No-ROX kit (Bioline, Memphis, Tenn., USA) on a fluorometric thermal cycler, Light Cycler (Roche), and data were analyzed with Light Cycler 3 software (Roche). The standard curve was constructed from the plasmid DNA of prcs2-2e1-egfp. The values of transcripts measured using RT-qPCR were normalized to the pothos ivy 5.8S gene and presented relative to the level of the transcript in clone VD1.

Benzene and Chloroform Uptake by Transformed Pothos Ivy

Sterile plantlets of pothos ivy clones (1 g) were incubated in 40 mL volatile organic analysis (VOA) vials (Fisher Scientific, 14-823-213), closed with septum valves (Mininert®, Valco Instruments Co. Inc., 614163), and containing 5 mL half-strength Hoagland's solution (Caisson Labs, HOP01-10LT.1). Wild-type untransformed plantlets and no-plant controls were incubated in parallel with clone VD3 and each treatment was repeated in quadruplicate.

Benzene gas was injected into the vials using gas-tight glass syringes to achieve a headspace concentration of 1850±160 mg m⁻³, taking into account gas liquid partitioning by Henry's Law. The vials were incubated for 9 days with rotary shaking at 80 rpm. The concentration of benzene was determined by manually injecting 100 μL of the headspace into a GC-FID (flame ionization detector) (Perkin Elmer AutoSystem XL). Chromatographic parameters were: oven temperature 60° C., injector temperature 250° C., and detector temperature 250° C., 1.33 ml min⁻¹ nitrogen carrier gas, using a ResTek RTX-1 microcapillary column (ResTek, 10121).

Similarly, chloroform was introduced into VOA vials using gas-tight glass syringes from sealed aqueous dilutions of chloroform (Acros Organics, 423550010). Transformed plantlets, wild type (WT), and no plant controls were incubated in quadruplicate, and headspace samples taken for analysis of chloroform levels by gas chromatography with an electron capture detector (GC-ECD) (Perkin Elmer AutoSystem XL) with a VOCOL capillary column 60 m×0.53 mm (Sigma). Chromatographic parameters were detector temperature at 325° C., nitrogen carrier gas at 1.76 ml min⁻¹, with a 100 ml min⁻¹ split, the oven at 100° C., and the injection port at 300° C.

EGFP Fluorescence

The EGFP signal of epidermal cells of pothos leaf was observed by fluorescent microscopy using the LSM 5 PASCAL system (ZEISS). The EGFP signal was excited by blue light and a FITI filter was used to collect fluorescent light. Axiocam 503 mono camera and software ZEN 2.3 lite were used to capture pictures.

Data Analysis

Data were analyzed for statistical significance using ANOVA in Microsoft Excel software (Microsoft Excel 2016 MSO). When ANOVA analysis gave a significant difference, Fishers Least Significant Difference (LSD) method was performed to compare the means. Groupings differing by statistical significance (p<0.05) are labeled by letters in the figures.

Calculation of Biofilter Sizing

The biofilters of the disclosure are designed to the standard used by the AHAM for the rating of particulate biofilters, the ⅔ rule: i.e., the clean air delivery rate (CADR, ft³/min, or m³/h) should be greater than or equal to ⅔ times the room area, ft², assuming an 8 ft ceiling. This number is based on a model that assumes 80% reduction in particulates in a room with exchange with exterior spaces equal to one room volume air change per hour. Particulate filters are tested by the AHAM in a standard room containing 1008 ft³ (28.5 m³), with an 8 ft ceiling and an area of 126 ft² (11.7 m²). For this room, a filter that meets the ⅔ rule would have a CADR equal to ⅔ times 126 or 84 cfm (2.4 m³/min). This works out to 0.67 cfm CADR per ft².

To calculate the size and operating parameters of a GM plant biofilter a first order model was used, which assumes that the pollutant removal rate is a function of the pollutant concentration. The model was derived as follows. A mass balance across the biofilter can be expressed as

QCᵢₙ−QCₒᵤₜ=Sink,

where the Sink represents the rate of pollutant removal in the biofilter, μg h⁻¹, and

Q=air flow through biofilter, m³ h⁻¹

Cᵢₙ=concentration of air pollutant entering biofilter, μg m⁻³

Cₒᵤₜ=concentration of air pollutant leaving biofilter, μg m⁻³.

It was assumed that removal of the pollutant was first-order with respect to the pollutant concentration, proportional to the plant biomass, and that the biofilter was completely mixed with the concentration in the biofilter equal to the effluent concentration.

Sink=V_{f}KMₚCₒᵤₜ, where

V_{f}=volume of biofilter, m³

K=first-order kinetic rate constant, h⁻¹ (g plant biomass)⁻¹

Mₚ=plant biomass, g

Thus,

$$Q ( {C}_{in} - {C}_{out} ) = {V}_{f} {KM}_{p} {C}_{out}$$

$${C}_{out} = \frac{{QC}_{in}}{{V}_{f} {KM}_{p} + Q}$$

$$\frac{{C}_{out}}{{C}_{in}} = \frac{Q}{{V}_{f} {KM}_{p} + Q}$$

Defining

E=removal efficiency for one pass through the biofilter,

$$E = 1 - \frac{{C}_{out}}{{C}_{in}}$$

$$E = 1 - \frac{Q}{{V}_{f} {KM}_{p} + Q}$$

Thus, the time course of the concentration in a batch experiment is exponential for benzene and chloroform uptake by a transgenic plant of the disclosure, such as an exemplary pothos clone vD3.

Calculation of the First-Order Rate Constants

In the biofilter sizing calculations, the uptake of chloroform rather than benzene was chosen, because benzene is oxidized by 2E1 to phenol, which may be toxic to the plants at the concentrations used for these experiments. Chloroform is almost immediately oxidized to nontoxic products CO₂ and chloride ion; therefore, it was believed that the rates for chloroform are more representative of those that are seen for both pollutants at home levels.

The level of 2e1 gene expression in the transformed pothos ivy is independent of benzene or chloroform concentration. Therefore, the kinetic parameters of pollutant degradation were expected to be invariant with pollutant concentration.

Biofilter Design Using an Exemplary Pothos Clone VD3

The rates for pollutant uptake have been determined in 40 mL vials containing plants weighing about 1 g. The first order rates for chloroform uptake by VD3 pothos are shown in Table 2. These rates were used for an exemplary biofilter design. This was a conservative assumption since the uptake rate in a forced air biofilter is likely to be greater than that in the 40 mL vials due to convective flow and turbulence in the biofilter. The first order rate constant for chloroform removal by exemplary pothos clone VD3 was 0.522 d⁻¹ (g biomass)⁻¹ (as shown in Table 2).

TABLE 2
Normalization of first-order kinetic constant for chloroform uptake by genetically
modified and wild-type pothos ivy plants to plant biomass and leaf area

| | First-order kinetic | Average plant | First-order kinetic constant, |
|---|---|---|---|
| Pothos ivy | constant, d⁻¹ | fresh biomass, g | d⁻¹ (g biomass)⁻¹ |
| 2E1 clone VD3 | −0.578 | 1.11 | −0.522 |
| Wild type | −0.026* | 1.08 | −0.024* |

*The slope of the semi-log plot of the wild-type time course of chloroform uptake was not significantly different from zero (p = 0.22).

If an air flow rate for the biofilter equal to 150 cfm (333 m³/h) and a plant mass of 11 kg (24 lb) is chosen, the design CADR of 84 cfm can be achieved with a biofilter volume of 1 m³ (35.3 ft²). This volume fills a double hung sash window of about 25 inches deep. Normalized to the area of the room and assuming a plant density in the biofilter of 11 kg/m³ (0.7 lb/ft³), it would take a biofilter of 0.052 m³ (1.8 ft³) with a plant biomass of 0.57 kg (1.25 lb) to provide 0.67 cfm CADR per ft² or 0.91 kg plant biomass per m².

Pothos has about 29 cm² of leaf area per g biomass, therefore 11 kg of plants have a leaf area of 32 m². An average pothos leaf grown indoors has an area of about 64 cm² per leaf (2 sides). A mature pothos growing indoors will grow to about 10 m, with leaves about every 2 cm, so there are about 500 leaves per mature plant and a total surface area of 3.2 m² per plant and about 1.1 kg of biomass per mature plant. Thus, 11 kg plant biomass equals to about 10 mature pothos plants, a number that can be easily achieved in an exemplary biofilter, e.g., a green wall.

### Results

Vector Construction and Generation of Transgenic Pothos Ivy

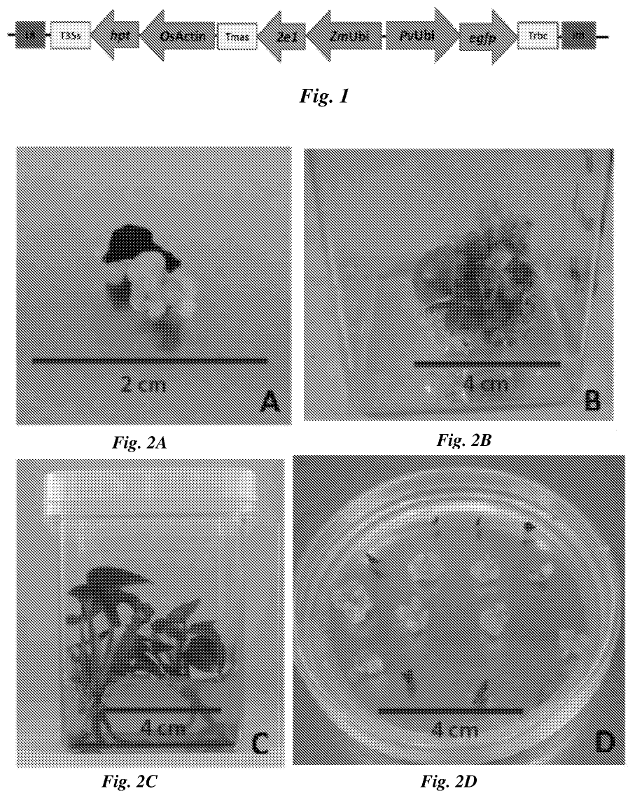

The structure of the plasmid pRCS2-2E1-EGFP used to transform pothos ivy is shown in FIG. 1. In order to achieve constant, high levels of expression, all of the transgenes were driven by constitutive monocot promoters. The hygromycin resistance gene, hpt, was driven by the actin promoter from rice (Oryza sativa) (McElroy, D. et al, Isolation of an efficient actin promoter for use in rice transformation. The Plant cell 1990, 2, (2), 163-71), the 2e1 gene was driven by the ubiquitin promoter of corn (Zea mays) (Cornejo, M. J.; et al., Activity of a maize ubiquitin promoter in transgenic rice. Plant molecular biology 1993, 23, (3), 567-81), and the egfp gene was driven by the ubiquitin promoter from switchgrass (Panicum virgatum) (Mann, D. G.; et al., Gateway-compatible vectors for high-throughput gene functional analysis in switchgrass (Panicum virgatum L.) and other monocot species. Plant biotechnology journal 2012, 10, (2), 226-36). The explants of pothos ivy were infected with EHA105 containing the vector pRCS2-2E1-EGFP and then screened on callus induction medium with hygromycin as selection agent for 2-3 months. Capitate somatic embryos developed from cut edges of leaf discs and petiole fragments (FIG. 2A). During subsequent culture, calli formed at the base and more cluster somatic embryos developed from the calli. After 3-4 months culture, the hygromycin-resistant calli were transferred to regeneration medium for induction of plantlets. After another 2-3 months culture, plantlets developed with both shoots and roots from the somatic embryos. Some plantlets developed only with shoots (FIG. 2B). These plants were transferred to MS medium with hygromycin for further growth and rooting (FIG. 2C). The leaf discs of PCR and RT-qPCR positive lines were cultured on E medium with 15 mg L⁻¹ hygromycin to induce somatic embryos for propagation while still under selection (FIG. 2D).

Molecular Analysis to Confirm the Transformation of Hygromycin-Resistant Lines

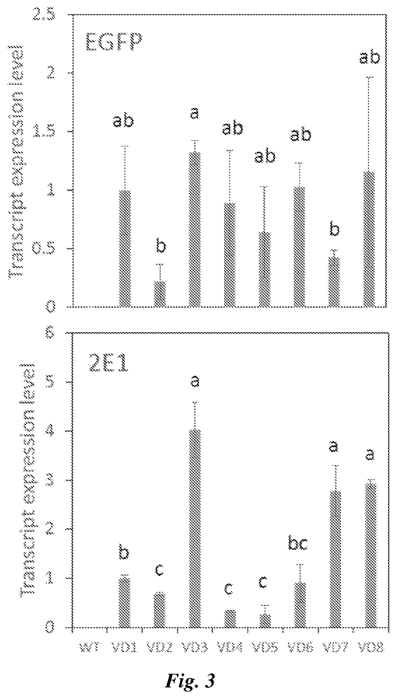

PCR primed by primer pairs annealing to promoter and terminator regions of 2e1 and egfp cassettes confirmed the integration of target genes into the genome of pothos ivy (data not shown). To measure the transcript abundance of 2e1 and egfp genes RT-qPCR was performed for eight transgenic lines, VD1-VD8. The expression levels of egfp were lower than that of 2e1, and were separated into two groups, with significant differences between VD3 and VD2 or VD7 (p<0.01, FIG. 3). The expression levels of the 2e1 gene between different transformed lines were different with high significance (p=0.00001). The clonal lines VD3, VD7, and VD8 had much higher expression levels of 2e1 compared to other lines. None of the transformed clonal lines had observable changes in morphology or growth compared to wild types.

EGFP Observation

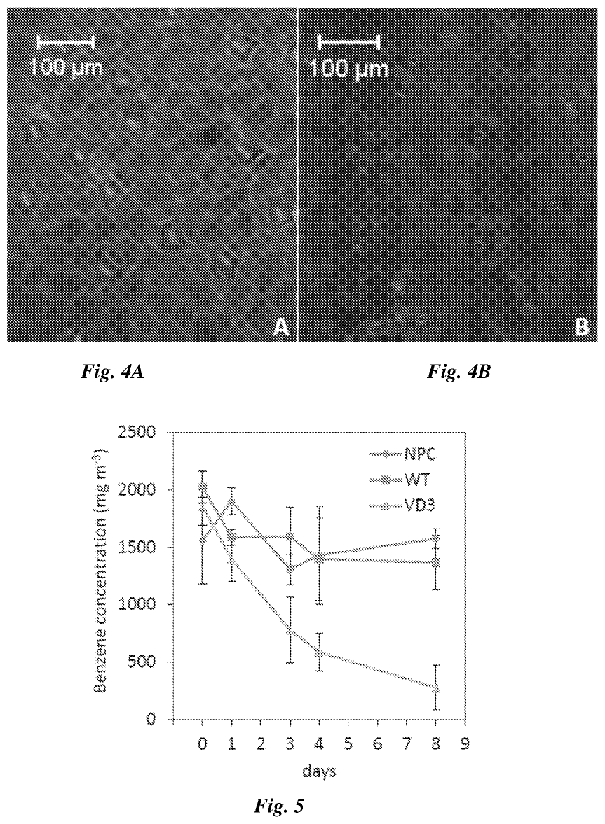

Using a fluorescent microscope, EGFP fluorescence was observed near the plasma membrane and around the nucleus (FIG. 4A) due to the presence of vacuoles in the epidermal cells of pothos ivy leaf. The emissions were marginally greater than emissions from the wild type, but weak. The wild-type cells were weakly autofluorescent generally, but not specifically from the cytosol. Green fluorescence was not visible to the eye in the transformed pothos ivy with handheld UV lamp illumination.

Benzene Uptake by Transformed Pothos Ivy

To determine the ability of 2e1-egfp transformed pothos ivy to take up benzene, plants were incubated in closed vials with the VOC. Benzene (144 μg) was injected into 40-mL VOA vials containing transformed and wild-type pothos ivy to achieve a final headspace concentration of 2500 mg m⁻³ benzene. After three days culture, the benzene concentration in vials with exemplary VD3 plants had fallen dramatically (FIG. 5). After eight days, the benzene concentration in no-plant vials had fallen by about 10%. The benzene concentrations in the vials containing exemplary VD3 plants were significantly different compared to vials containing wild-type plants after three days culture (p=0.039), p=0.012 at day 4, and p=0.0008 at day 8.

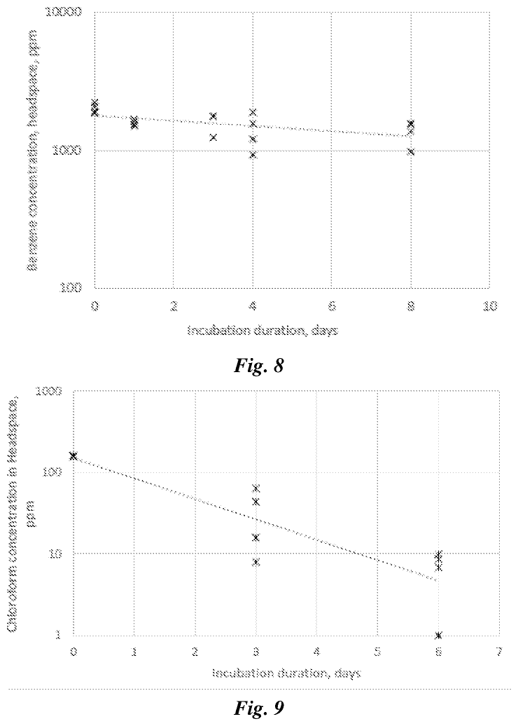

The time course of the benzene concentration in the vials with exemplary transformed pothos ivy clone VD3 was plotted on semi-logarithmic axes and fit by linear regression with a first-order rate constant equal to (−0.249 d⁻¹, FIG. 7), or −0.115 d⁻¹ (g fresh biomass)⁻¹. Since small pothos plants have 29 cm² leaf area (g fresh biomass)⁻¹, this kinetic constant is equivalent to −39.8 d⁻¹ (m² leaf area)⁻¹, normalized to leaf area. The slope of the best linear fit to the semi-logarithmic plot of the time course of benzene concentration for wild-type plants (−0.044 d⁻¹, FIG. 8) was significantly different from zero (p=0.015), suggesting that the wild-type plants did take up some benzene. The wild-type pothos took up benzene at a first-order rate normalized to biomass equivalent to −0.024 d⁻¹ (g biomass)⁻¹, or −8.5 d⁻¹ (m² leaf area)⁻¹, normalized to leaf area. The normalized rate constant for uptake of benzene uptake by exemplary transformed clone VD3 was 4.7 times that of the wild-type.

Chloroform Uptake by Exemplary Transformed Pothos Ivy

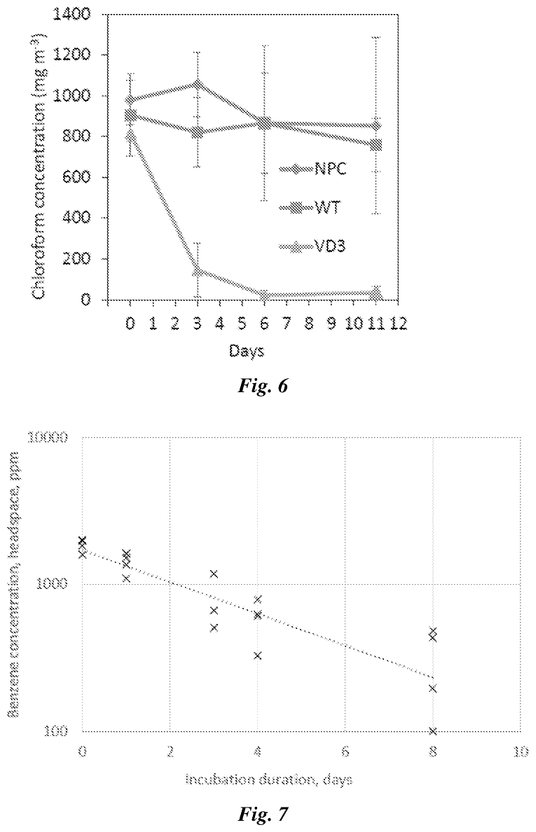

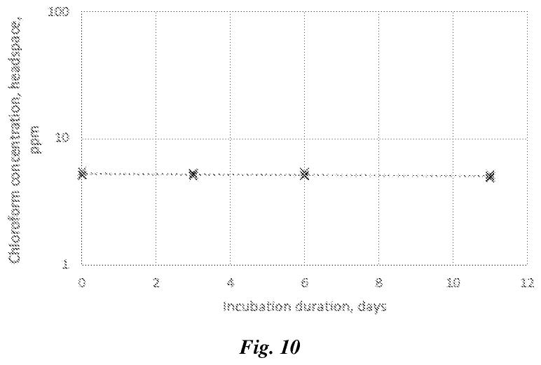

The concentration of chloroform in the headspace of vials incubated with exemplary VD3 plants fell rapidly, while chloroform concentrations in incubations with wild-type plantlets and no-plant controls did not change significantly (FIG. 6). The concentration of chloroform decreased by 82% during the first 3 days in the vials containing exemplary clone VD3 plants and chloroform was barely detectable after 6 days. Linear regression of the semi-logarithmic plot of the chloroform data yielded a first-order degradation constant equal to −0.549 d⁻¹ (FIG. 9). The slope of the best linear fit to the semi-logarithmic plot of the time course of chloroform concentration for wild-type plants (FIG. 10) was not significantly different from zero (p=0.22), suggesting that the wild-type plants did not take up chloroform. The rate constant for the exemplary VD3 transformed pothos ivy normalized to biomass was 0.552 d⁻¹ (g fresh biomass)⁻¹, equivalent to 180 d⁻¹ (m² leaf area)⁻¹, normalized to leaf area.

### Discussion of the Results

VOCs in indoor air pose significant cancer risks to vulnerable populations, such as children, yet there are no practical, sustainable technologies available for their removal in the home. Physical-chemical methods based on sorbents and oxidation methods are energy intensive and of limited use for the removal of formaldehyde and chloroform, respectively. Various houseplants have been touted as having the ability to remove VOCs from air, but plant uptake rates vary exponentially from one study to another. Many studies appear to be affected by artifactual enhancement of soil bacterial activities by high VOC concentrations. Herein, high VOC concentrations were used to facilitate analysis by hand injection of headspace samples onto GC-FID in the case of benzene, but the assays were performed in axenic conditions, without bacterial activity. As can be seen in FIGS. 5 and 6, there was little or no loss of benzene or chloroform in the vials containing wild-type pothos ivy, while most of the benzene and all of the chloroform was removed in 6 days in the vials with 2E1-expressing exemplary clone VD3. These results show the effectiveness of genetically modified pothos for VOC removal compared to wild-type pothos.

Expression of green fluorescent protein was intended as a visible indication that the pothos ivy was transformed, but fluorescence of transformed clone VD3 was too weak to be visible without microscopy. Other variants of GFP, such as mGFP-ER and the use of a stronger monocot promoter can provide stronger fluorescence.

Removal of VOCs from home air using transgenic houseplants described herein can be made by combining expression of 2e1 with other detoxifying genes. Formaldehyde, the other VOC that poses most risk in home air is of prime interest. Overexpression of faldh gene from Brevibacillus brevis in tobacco conferred plants a high tolerance to HCHO and increased the ability to take up formaldehyde 2-3 times faster than wild-type plants. The faldh gene can be stacked with 2e1 and other detoxifying genes in vectors that are used to genetically modify pothos ivy, resulting in plants that could degrade most of the important indoor air VOCs. Thus, in certain embodiments of the transgenic houseplant disclosed herein, the expression cassette further comprises a polynucleotide sequence encoding an aldehyde dehydrogenase (ALDH), such as FALDH or ALDH1a1.

Thus, in some embodiments, the transgenic houseplant disclosed herein comprises a polynucleotide sequence SEQ ID NO 16 or 18. In some embodiments, the aldehyde dehydrogenase comprises a peptide sequence of SEQ ID NO: 17 or 19.

Since 2e1 gene expression in the transformed pothos ivy is under constitutive promoters, the level of 2E1 expression is independent of benzene or chloroform concentration. Therefore, the kinetic parameters of pollutant degradation are expected to be invariant with pollutant concentration.

Performance of an enclosed, forced-air biofilter was calculated empirically using the same first-order degradation constant observed in the batch experiments with chloroform, 0.52 d⁻¹ (g biomass)⁻¹. For the case of a completely-mixed biofilter with a volume of 0.7 m³ and an airflow rate of 300 m³ h⁻¹, about 10 kg of exemplary pothos ivy clone VD3 can remove 34% of the chloroform in one pass. This biofilter can have a clean air delivery rate (CADR) of 100 m³ h⁻¹, comparable to CADRs of current commercial home particulate filters. This calculation demonstrates that genetically modified plants, such as the transgenic houseplants disclosed herein, have practical utility for sustainable phytoremediation of home air.

Compared to current chemical/physical methods for removal of VOCs from indoor air, biofilters using transgenic plants offer the advantages of low energy use and decreased need for maintenance. All of the removal methods require a means for moving the air through the apparatus, however, unlike biofilters disclosed herein, adsorptive methods require significant energy expenditure to regenerate the media, and photooxidative methods require high energy inputs to oxidize the pollutants, making those methods less sustainable. Transgenic phytoremediation requires very little additional energy beyond that required for air movement. Pothos ivy is well adapted to medium- and low-light levels so artificial lighting would usually not be required, giving phytoremediation an intrinsic sustainability advantage.

For convenience, certain terms employed in the specification, examples, and appended claims are provided here. The definitions are provided to aid in describing particular embodiments and are not intended to limit the claimed invention, as the scope of the invention is limited only by the claims.

The use of the term “or” in the claims and specification is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an open and inclusive sense as opposed to a closed, exclusive or exhaustive sense. For example, the term “comprising” can be read to indicate “including, but not limited to.” The term “consists essentially of” or grammatical variants thereof indicate that the recited subject matter can include additional elements not recited in the claim, but which do not materially affect the basic and novel characteristics of the claimed subject matter.

As used herein, the word “about” indicates a number within range of minor variation above or below the stated reference number. For example, “about” can refer to a number within a range of 10% above or below the indicated reference number. As used herein, “plant parts” refer to tissues, organs, seeds, and severed parts (e.g., cuttings) that retain the distinguishing characteristics of the parent plant.

While illustrative embodiments have been illustrated and described, it will be appreciated that various changes can be made therein without departing from the spirit and scope of the invention.

1. A transgenic houseplant, houseplant part, or houseplant cell stably transformed with an expression cassette comprising a first polynucleotide sequence encoding a mammalian cytochrome protein, wherein the transgenic houseplant is capable of removing a volatile organic compound from air.

2. The transgenic houseplant, houseplant part, or houseplant cell of claim 1, wherein the expression cassette further comprises a second polynucleotide sequence encoding a visual selection marker.

3. The transgenic houseplant, houseplant part, or houseplant cell of claim 1, wherein the first polynucleotide sequence is operably linked to a first promoter functional in the transgenic houseplant.

4. The transgenic houseplant, houseplant part, or houseplant cell of claim 2, wherein second polynucleotide sequence is operably linked to a second promoter functional in the transgenic houseplant.

5. The transgenic houseplant, houseplant part, or houseplant cell of claim 1, wherein the transgenic houseplant is transgenic Epipremnum aureum.

6. The transgenic houseplant, houseplant part, or houseplant cell of claim 1, wherein the mammalian cytochrome protein is Cytochrome P450 or a variant or a homolog thereof with at least about 90% amino acid sequence homologous thereto.

7-8. (canceled)

9. The transgenic houseplant, houseplant part, or houseplant cell of claim 1, wherein the first polynucleotide sequence encoding the mammalian cytochrome protein is codon-optimized for plants.

10. (canceled)

11. The transgenic houseplant, houseplant part, or houseplant cell of claim 2, wherein the visual selection marker is a fluorescent protein.

12. The transgenic houseplant, houseplant part, or houseplant cell of claim 11, wherein the fluorescent protein is green fluorescent protein mGFP-ER, eGFP, or a variant or a homolog thereof with at least about 90% amino acid sequence homologous thereto.

13. The transgenic houseplant, houseplant part, or houseplant cell of claim 2, wherein the expression cassette further comprises a third polynucleotide sequence encoding a positive selection marker.

14. The transgenic houseplant, houseplant part, or houseplant cell of claim 13, wherein the positive selection marker is a protein conferring antibiotic resistance to the transgenic houseplant.

15. The transgenic houseplant, houseplant part, or houseplant cell of claim 14, wherein the positive selection marker is Hygromycin B phosphotransferase or a variant or a homolog thereof with at least about 90% amino acid sequence homologous thereto.

16. The transgenic houseplant, houseplant part, or houseplant cell of claim 13, wherein the expression cassette further comprises a fourth polynucleotide sequence encoding an aldehyde dehydrogenase enzyme.

17. The transgenic houseplant, houseplant part, or houseplant cell of claim 16, wherein the aldehyde dehydrogenase is a formaldehyde dehydrogenase (FALDH) or a variant or a homolog thereof with at least about 90% amino acid sequence homologous thereto.

18. The transgenic houseplant, houseplant part, or houseplant cell of claim 1, wherein the volatile organic compound is selected from the group consisting of 1,4-dichlorobenzene, benzene, 1,3-butadiene, formaldehyde, acetaldehyde, naphthalene, acrylonitrile, carbon tetrachloride, dichlorobromomethane, dibromochloromethane, bromoform, trichloroethylene, vinyl chloride, methyl chloroform, cis-1,2-dichloroethylene, chloroform, and combinations thereof.

19. A method of reducing a concentration of a volatile organic compound in air comprising contacting a transgenic houseplant of claim 1 with air comprising a volatile organic compound vapor.

20-21. (canceled)

22. An air purifying biofilter comprising: (a) a planting structure comprising one or more transgenic houseplants of claim 1 and (b) an air flow device coupled to the planting structure and adapted to maintain airflow around the plants.

23. The air purifying biofilter of claim 22, wherein the air flow device increases mass air flow over the leaf surfaces of the one or more transgenic houseplants.

24-25. (canceled)

26. The air purifying biofilter of claim 22, wherein the biofilter is capable of about 80% or greater reduction in particulate in a room with air exchange with exterior spaces equal to one room volume air change per hour.

27. (canceled)

28. The air purifying biofilter of claim 22, wherein the air purifying biofilter comprises an enclosure surrounding the one or more transgenic houseplants.

29-30. (canceled)
